(19) United States
(12) Patent Application Publication
(10) Pub. No.: US 2006/0183922 A1
(43) Pub. Date: Aug. 17, 2006

(54) CB2-selective cannabinoid derivatives

(76) Inventors: Billy R. Martin, Richmond, VA (US); John William Huffman, Dillsboro, NC (US); Mohamad Imad Damaj, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/155,118

(22) Filed: Jun. 17, 2005

Related U.S. Application Data
(60) Provisional application No. 60/653,511, filed on Feb. 17, 2005.
(60) Provisional application No. 60/659,091, filed on Mar. 8, 2005.
(60) Provisional application No. 60/663,210, filed on Mar. 21, 2005.

(51) Int. Cl. C07D 311/80

(52) U.S. Cl. 549/390

(57) ABSTRACT

Cannabinoid derivatives that exhibit specificity for the CB₂ cannabinoid receptor are provided. The analogues are tetrahydrocannabinols and hydroxyhexahydrocannabinols, and are useful for the treatment of pain, inflammation, and cancer.

### CROSS-REFERENCE TO RELATED APPLICATIONS

This application claims benefit of U.S. provisional patent application 60/653,511, filed Feb. 17, 2005; U.S. provisional patent application 60/659,091, filed Mar. 8, 2005; and U.S. provisional patent application 60/663,210, filed Mar. 21, 2005, the compete contents of each of which is hereby incorporated by reference.

This invention was made using funds from grants from the National Institutes of Health having grant numbers DA 03672 and DA 03590. The United States government may have certain rights in this invention.

### BACKGROUND OF THE INVENTION

1. Field of the Invention

The invention generally relates to cannabinoid derivatives. In particular, the invention provides cannabinoid derivatives that exhibit a high level of specificity for the CB₂ cannabinoid receptor.

2. Background of the Invention

The complex effects of cannabinoids are considered to be mediated through at least two G-protein coupled, transmembrane receptors. One of these, designated as CB₁, is found predominantly in the central nervous system and is responsible for most of the overt pharmacological effects of cannabinoids. A second receptor, designated CB₂ was originally identified from macrophages present in the spleen, and is almost exclusively found in tissues of the immune system such as the spleen, tonsil and lymph nodes. Although it has been known for some time that cannabinoids are involved in immunomodulation, the discovery that CB₂ receptors are expressed primarily in the immune system led to the suggestion that the CB₂ receptor was responsible for the immunomodulatory effects of cannabinoids. This suggestion was confirmed by the observation that such effects are absent in CB₂ receptor knockout mice. Furthermore, other studies have demonstrated other beneficial effects specific to the CB₂ receptor, such as involvement in anti-inflammatory processes, and in the prevention of tumor cell growth. Another advantage of developing compounds specific for the CB₂ receptor is that such compounds lack the psychotropic side effects exhibited by compounds that interact with the CB, receptor.

There is thus an ongoing need for the development of ligands that are specific or selective for the CB₂ receptor.

### SUMMARY OF THE INVENTION

It is an object of this invention to provide cannabinoid derivatives that bind preferentially to the CB₂ cannabinoid receptor, and which exhibit little or no binding affinity for the CB₁ receptor. The derivatives are tetrahydrocannabinols and hydroxyhexahydrocannabinols. The derivatives can be used for the treatment of CB₂ receptor mediated conditions, diseases or disorders such as pain, inflammation, and cancer. Compounds that are selective for the CB₂ receptor are advantageous in that they do not induce the psychotropic side effects exhibited by compounds that interact with the CB₁ receptor.

The present invention provides a compound of general formula 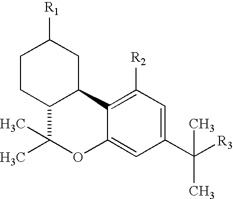
in which R₁ is OH and may be in either the α or β configuration; R₂ may be H or OCH₃; and R₃ is an alkyl chain from 2 to 6 carbon atoms in length. Particular examples of this compound include:

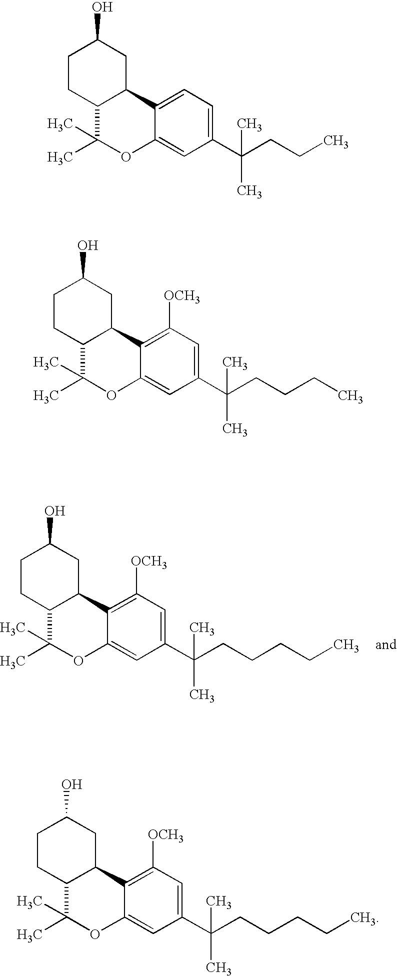

The invention further provides compounds of the general formula 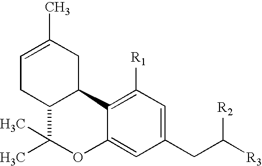
in which R₁ is H or OCH₃; R₂ is CH₃ and is in either the R or S configuration; and R₃ is an alkyl chain from 2 to 6 carbon atoms in length. Examples of this type of compound include

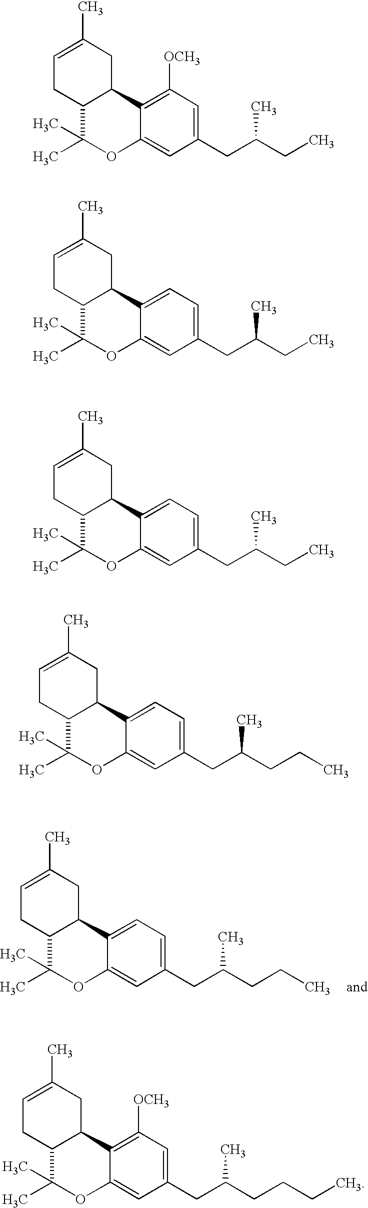

The invention further provides a method for selectively binding to CB₂ receptors and not to CB₁ receptors. The method comprises the step of exposing the CB₂ receptors and the CB₁ receptors to a compound of general formula 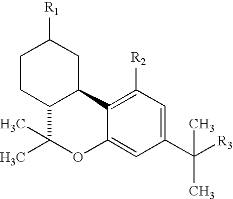
in which R₁ is OH and may be in either the α or β configuration; R₂ may be H or OCH₃; and R₃ is an alkyl chain from 2 to 6 carbon atoms in length. Examples of such a compound are as follows 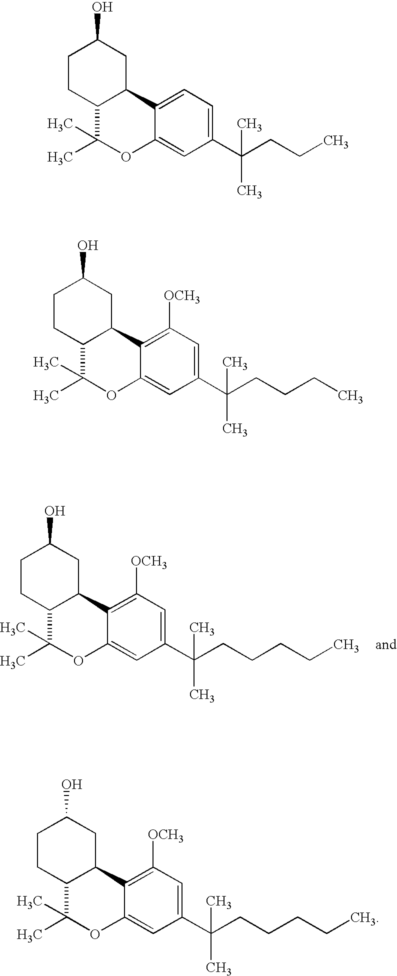
In one embodiment of the invention, the compound binds to the CB₂ receptors in an amount sufficient to treat cancer. In another embodiment of the invention, the compound binds to the CB₂ receptors in an amount sufficient to treat pain. In yet another embodiment of the invention, the compound binds to the CB₂ receptors in an amount sufficient to treat inflammation.

The present invention also provides a method for selectively binding to CB₂ receptors and not to CB₁ receptors. The method comprises the step of exposing the CB₂ receptors and the CB₁ receptors to a compound of general formula 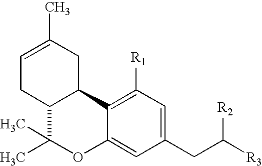
wherein R₁ is H or OCH₃; R₂ is CH₃ and is in either the R or S configuration; and R₃ is an alkyl chain from 2 to 6 carbon atoms in length.
Examples of such compounds include: 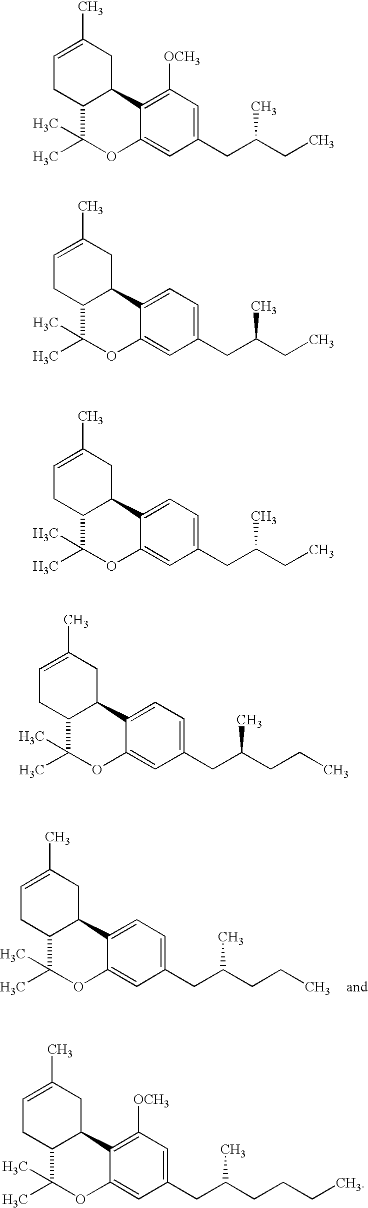
In one embodiment of the invention, the compound binds to the CB₂ receptors in an amount sufficient to treat cancer. In another embodiment of the invention, the compound binds to the CB₂ receptors in an amount sufficient to treat pain. In yet another embodiment of the invention, the compound binds to the CB₂ receptors in an amount sufficient to treat inflammation.

### DETAILED DESCRIPTION OF THE DRAWINGS

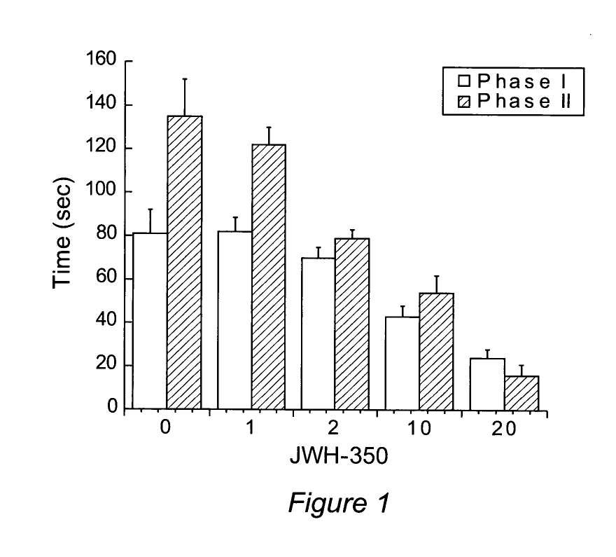

FIG. 1. Inhibition of inflammatory pain: formalin model. 20 mg/kg of compound JWH-350 completely inhibited pain response.

### DETAILED DESCRIPTION OF THE PREFERRED EMBODIMENTS OF THE INVENTION

The present invention provides cannabinoid derivatives that bind with high affinity to the CB₂ cannabinoid receptor and methods for their use in the treatment of various diseases and disorders. By binding with “high affinity” we mean that the Kᵢ values of the compounds for the CB₂ receptor are in the range of about 0.03 nM to about 50 nM. In a preferred embodiment, the compounds display both high binding affinity for the CB₂ receptor and high binding selectivity for the CB₂ receptor. By “binding selectivity” we mean that the compounds exhibit an affinity for the CB₂ cannabinoid receptor that is in the range of about 10 to about 1000 fold or more higher than their affinity for the CB₁ cannabinoid receptor. In other words, the Kᵢ values of the compounds for CB₂ receptors are in the range of about 10 to about 1000 times (or more) lower than the Kᵢ values of the compounds for the CB₁ receptor. Measurement of the Kᵢ values may be carried out in any of several known assay systems, including but not limited to biological tissue such as brain and spleen, or in cells that are genetically modified to express CB₁ or CB₂ receptors. In a preferred embodiment, the assay system that is utilized is brain tissue that contains CB₁ receptors and cells genetically modified to express CB₂ receptors.

In one embodiment of the invention, the compounds are of the general formula represented in Formula 1 below.

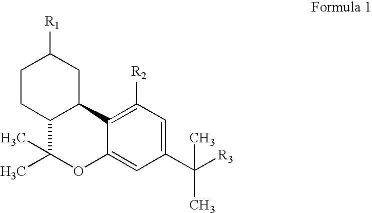

In the compounds of generic Formula 1, R₁ is OH and may be in either the α or β configuration around carbon 9; R₂ may be H or OCH₃; and R₃ is an alkyl chain from about 2 to about 6 carbon atoms in length.

In preferred embodiments of the invention, the compound of Formula 1 is one of the following:

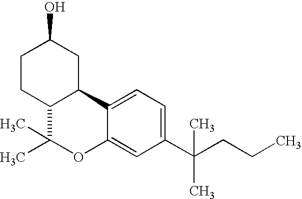

### 1-Deoxy-9β-hydroxy-3-(1′,1′dimethylbutyl)-11-nor-hexahydrocannabinol (JWH-310)

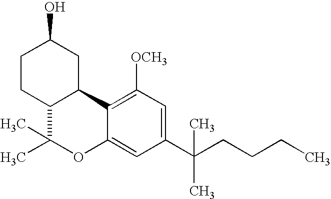

### 1-Deoxy-9β-Hydroxy-1-methoxy-3-(1′,1′dimethylpentyl)-11-nor-hexahydrocannabinol (JWH-299)

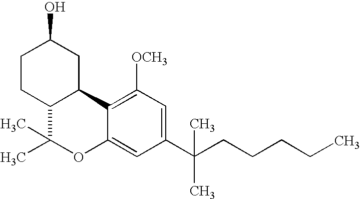

### 9β-Hydroxy-1-methoxy-3-(1′,1′dimethylhexyl)-11-nor-hexahydrocannabinol (JWH-350)

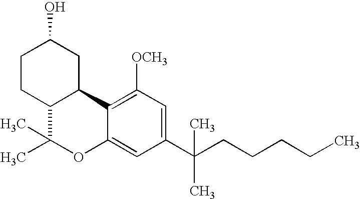

### 9α-Hydroxy-1-methoxy-3-(1′,1′dimethylhexyl)-11-nor-hexahydrocannabinol (JWH-349)

In another embodiment of the invention, the compounds of the invention are of the general formula represented in Formula 2 below.

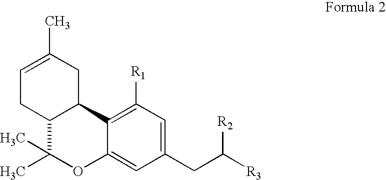

In the compounds of general Formula 2, R₁ is H or OCH₃; R₂ is CH₃ and is in either the R or S configuration, and R₃ is an alkyl chain from about 2 to about 6 carbon atoms in length. In preferred embodiments of the invention, the compound of Formula 2 is one of the following:

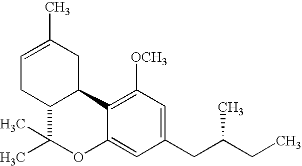

### 1-Methoxy-3-(2′R-methylbutyl)-Δ⁸-tetrahydrocannabinol (JWH-359)

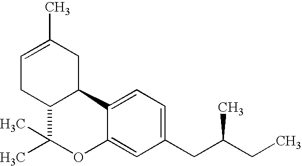

### 1-Deoxy-3-(2′S-methylbutyl)-Δ⁸-tetrahydrocannabinol (JWH-352)

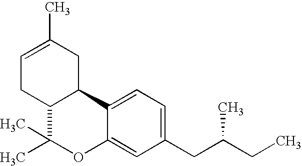

### 1-Deoxy-3-(2′R-methylpentyl)-Δ⁸-tetrahydrocannabinol (JWH-353)

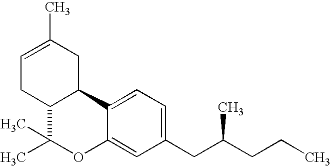

### 1-Deoxy-3-(2′S-methylpentyl)-Δ⁸-tetrahydrocannabinol (JWH-255)

and

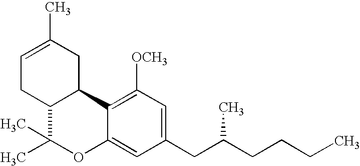

### 1-Methoxy-3-(2′R-methylhexyl)-Δ⁸-tetrahydrocannabinol (JWH-356)

The compounds of the present invention are useful for a variety of therapeutic applications. For example, they may be used to treat pain (i.e. as analgesics) in a variety of applications including but not limited to pain management. Pain of any type may be ameliorated or managed by the administration of the compounds of the present invention. Examples include but are not limited to: pain related acute but temporary conditions (e.g. due to injury, surgical procedures, etc.); and pain related to chronic conditions (for example, ongoing, chronic pain caused by injuries such as neck/spinal injuries, athletic injuries, etc., or disease, e.g. pain caused by cancer, shingles, arthritis, etc.). The compounds of the present invention may be used to treat (e.g. to prevent, lessen or eliminate) pain due to any cause.

The compounds of the present invention are also useful for the treatment of diseases or disorders related to or caused by inflammation. The inflammation may be either acute (e.g., inflammation from injuries or from surgery) or chronic (e.g. inflammation from autoimmune disorders such as arthritis, terminal cancer, etc.).

In addition, the compounds of the present invention may be used for the treatment of cancer. Examples of types of cancer that may be treated by the administration of the present compounds include but are not limited to glioma tumors and non-melanoma skin cancers. In a preferred embodiment, the compounds are used to treat glioma tumors. Those of skill in the art will recognize that CB₂ cannabinoid receptor agonists are known to exhibit efficacy in causing regression of tumors (e.g. see Sanchez, J, et al. Cancer Research 2001, 61, 5784-5789; Casanova, M. L. et al. J. Clin. Invest. 2003, 111, 43-50; and Blazquez, C. et al. FASEB J. 2003, 17, 529-531).

The invention thus provides methods for treating pain, inflammation and/or cancer in a patient in need thereof. By “treating” we mean that the compound is administered in order to alleviate symptoms of the disease or disorder being treated. Those of skill in the art will recognize that the symptoms of the disease or disorder that is treated may be completely eliminated, or may simply be lessened. For example, in the treatment of cancer, administration of the compounds of the present invention may completely eradicate symptoms of the disease, or, alternatively, may attenuate or slow the progression of disease (e.g. by decreasing tumor size, halting metastasis, etc.), which is also beneficial to the patient. Further, the compounds may be administered in combination with other drugs or treatment modalities.

In a more general sense, the invention also provides methods of treating conditions or disorders related to CB₂ receptor-regulated systems. By “CB₂ receptor-regulated systems” we mean biochemical pathways that include the binding of a cannabinoid to the CB₂ receptor molecule within the pathway. Examples of such biochemical pathways include but are not limited to activation of G-proteins and subsequent inhibition of adenylyl cyclase. The compounds of the present invention may be used to treat aberrations in CB₂ receptor-regulated systems, or to alter CB₂ receptor-regulated systems when it is desirable to do so in order to ease untoward symptoms in a patient. For example, patients with pain or spasticity arising from inflammation or neuropathological damage will benefit from activation of the CB₂ receptor with a selective agonist. In addition, the compounds may also be used for research purposes.

Implementation will generally involve identifying patients suffering from the indicated disorders and administering the compounds of the present invention in an acceptable form by an appropriate route. The exact dosage to be administered may vary depending on the age, gender, weight and overall health status of the individual patient, as well as the precise etiology of the disease. However, in general for administration in mammals (e.g. humans), dosages in the range of from about 0.1 to about 30 mg of compound per kg of body weight per 24 hr., and more preferably about 0.1 to about 10 mg of compound per kg of body weight per 24 hr., are effective.

Administration may be oral or parenteral, including intravenously, intramuscularly, subcutaneously, intradermal injection, intraperitoneal injection, etc., or by other routes (e.g. transdermal, sublingual, oral, rectal and buccal delivery, inhalation of an aerosol, etc.).

The compounds may be administered in the pure form or in a pharmaceutically acceptable formulation including suitable elixirs, binders, and the like (generally referred to a “carriers”) or as pharmaceutically acceptable salts (e.g. alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil based vehicles. Water may be used as the carrier for the preparation of compositions (e.g. injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc). Solid diluents and excipients include lactose, starch, conventional disintergrating agents, coatings and the like. Preservatives such as methyl paraben or benzalkium chloride may also be used. Depending on the formulation, it is expected that the active composition will consist of about 1% to about 99% of the composition and the vehicular “carrier” will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, or at a gradual or continuous, constant or controlled rate to a patient. In addition, the time of day and the number of times per day that the pharmaceutical formulation is administered may vary are and best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities. The compounds may also be added to blood ex vivo and then be provided to the patient.

### EXAMPLES

It has been established that a 1′,1′-or 1′,2′-dimethylheptyl side chain greatly enhances CB1 receptor affinity and cannabinoid potency. Similarly, a 1′- ro 2′-methyoheptyl group also increases CB1 receptor affinity and potency (Huffman et al., Biorg. Med. Chem. 1998, 6, 2383). In both the 1′,1′-dimethylalkyl-Δ⁸-THC series and the homologous series of Δ⁸-THC analogs with an unsubstituted side chain, those cannabinoids with a side chain of five to eight carbon atoms are most potent (Huffman et al., Biorg. Med. Chem. 2003, 11, 1397). In a series of 1-methoxy- and 1-deoxy-Δ⁸-THC analogs, it was found that CB₂ receptor affinity is also enhanced by the presence of a 1′,1′-dimethylalkyl side chain. However, CB₂ receptor affinity is much less sensitive to chain length. For instance, the three carbon analog, 3-(1′,1′-dimethylpropyl)-1-deoxy-Δ⁸-THC has Kᵢ=14 nM for the CB₂ receptor, and 1-deoxy-Δ⁸-THC-DMH with a seven carbon side chain has only slightly greater affinity with a Kᵢ=3 nM (Huffman et al., Biorg. Med. Chem. 1999, 76, 2005). Prior to the present investigations, nothing was known about the effect of side chain substituents other than a 1′,1′-dimethyl upon CB₂ receptor affinity. The present studies were undertaken to obtain additional data concerning structure-activity relationships at the CB₂ receptor and to investigate the influence of side chain stereochemistry upon receptor affinity.

### Example 1

### Synthesis of Exemplary Compounds of Formula 1

1-Deoxy-9β-hydroxy-3-(1′,1′dimethylbutyl)-11-nor-hexahydrocannabinol, JWH-310, shown below, is prepared from the mixture of acetates obtained by lead tetraacetate oxidation of nopinone and 2-methyl-2-(3,5,-dihydroxyphenyl)pentane which gives 9-keto-3-(1′.1′dimethylbutyl)-11-nor-hexahydrocannabinol. Conversion to the diethylphosphate ester, followed by dissolving metal reduction provides 1-deoxy-9β-hydroxy-3-(1′,1′dimethylbutyl)-11-nor-hexahydrocannabinol (JWH-310).

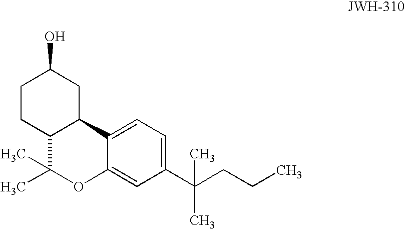

1-Deoxy-9β-hydroxy-1-methoxy-3-(1′,1′dimethylpentyl)-11-nor-hexahydrocannabinol, JWH-299, shown below, is prepared from the mixture of acetates obtained by lead tetraacetate oxidation of nopinone and 2-methyl-2-(3,5,-dihydroxyphenyl)hexane which gives 9-keto-3-(1′.1′dimethylpentyl)-11-nor-hexahydrocannabinol. Conversion to the methyl ether is effected by reaction with methyl iodide and potassium hydroxide to provide 9-keto-1-methoxy-3-(1′.1′dimethylpentyyl)-11-nor-hexahydrocannabinol. Stereoselective reduction with sodium borohydride gives 1-deoxy-9β-hydroxy-1-methoxy-3-(1′,1′dimethylpentyl)-11-nor-hexahydrocannabinol (JWH-299).

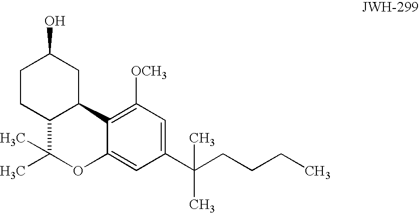

9β-Hydroxy-1-methoxy-3-(1′,1′dimethylhexyl)-11-nor-hexahydrocannabinol, JWH-350, shown below, is prepared from the mixture of acetates obtained by lead tetraacetate oxidation of nopinone and 2-methyl-2-(3,5,-dihydroxyphenyl)heptane which gives 9-keto-3-(1′.1′dimethylhexyl)-11-nor-hexahydrocannabinol. Conversion to the methyl ether is effected by reaction with methyl iodide and potassium hydroxide to provide 9-keto-1-methoxy-3-(1′.1′dimethylhexyl)-11-nor-hexahydrocannabinol. Stereoselective reduction with sodium borohydride gives 9β-hydroxy-1-methoxy-3-(1′,1′dimethylhexyl)-11-nor-hexahydrocannabinol (JWH-350).

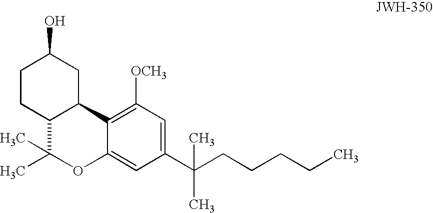

9α-Hydroxy-1-methoxy-3-(1′,1′dimethylhexyl)-11-nor-hexahydrocannabinol, JWH-349, shown below, is prepared from the mixture of acetates obtained by lead tetraacetate oxidation of nopinone and 2-methyl-2-(3,5,-dihydroxyphenyl)heptane which gives 9-keto-3-(1′.1′dimethylhexyl)-11-nor-hexahydrocannabinol. Conversion to the ether is effected by reaction with methyl iodide and potassium hydroxide to provide 9-keto-1-methoxy-3-(1′,1′dimethylhexyl)-11-nor-hexahydrocannabinol. Stereoselective reduction with L-selectride gives 9α-Hydroxy-1-methoxy-3-(1′,1′dimethylhexyl)-11-nor-hexahydrocannabinol (JWH-349).

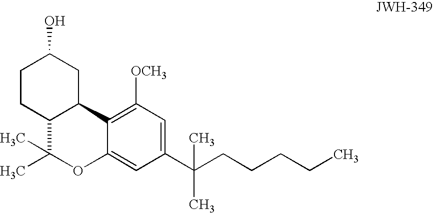

### Example 2

### Synthesis of Exemplary Compounds of Formula 2

1-Methoxy-3-(2′R-methylbutyl)-Δ⁸-tetrahydrocannabinol, JWH-359, shown below, is prepared from 2R-methyl-(3,5-dihydroxyphenyl)butane and trans-menthadienol to give 3-(2′R-methylbutyl)-Δ⁸-tetrahydrocannabinol. Conversion to the methyl ether is effected by reaction with methyl iodide and potassium hydroxide to provide 1-methoxy-3-(2′R-methylbutyl)-Δ⁸-tetrahydrocannabinol, JWH-359 (JWH-359). 2R-methyl-(3,5-dihydroxyphenyl)butane is prepared in several steps in an enantioselective synthesis from a D-valine derived oxazolidinone of propionic acid and 3,5-dimethoxybenzyl bromide.

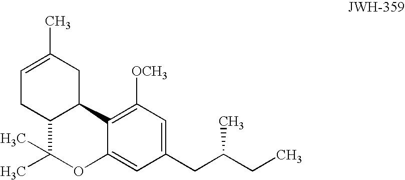

1-Deoxy-3-(2′S-methylbutyl)-Δ⁸-tetrahydrocannabinol, JWH-352, shown below, is prepared from 2S-methyl-(3,5-dihydroxyphenyl)butane and trans-menthadienol to give 3-(2′S-methylbutyl)-Δ⁸-tetrahydrocannabinol. Conversion to the diethylphosphate ester, followed by dissolving metal reduction provides 1-deoxy-3-(2′S-methylbutyl)-Δ⁸-tetrahydrocannabinol (JWH-352). 2S-methyl-(3,5-dihydroxyphenyl)butane is prepared in several steps in an enantioselective synthesis from an L-valine derived oxazolidinone of propionic acid and 3,5-dimethoxybenzyl bromide.

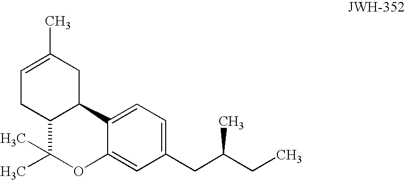

1-Deoxy-3-(2′R-methylbutyl)-Δ⁸-tetrahydrocannabinol, JWH-353, shown below, is prepared from 2R-methyl-(3,5-dihydroxyphenyl)butane and trans-menthadienol to give 3-(2′R-methylbutyl)-Δ⁸-tetrahydrocannabinol. Conversion to the diethylphosphate ester, followed by dissolving metal reduction provides 1-deoxy-3-(2′R-methylbutyl)-Δ⁸-tetrahydrocannabinol (JWH-353). 2R-methyl-(3,5-dihydroxyphenyl)butane is prepared in several steps in an enantioselective synthesis from a D-valine derived oxazolidinone of propionic acid and 3,5-dimethoxybenzyl bromide.

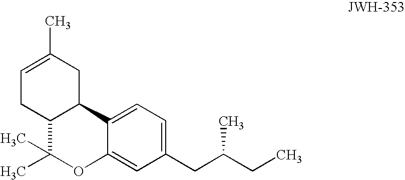

1-Deoxy-3-(2′S-methylpentyl)-Δ⁸-tetrahydrocannabinol, JWH-255, shown below, is prepared from 2S-methyl-(3,5-dihydroxyphenyl)pentane and trans-menthadienol to give 3-(2′S-methylpentyl)-Δ⁸-tetrahydrocannabinol. Conversion to the diethylphosphate ester, followed by dissolving metal reduction provides 1-deoxy-3-(2′S-methylpentyl)-Δ⁸-tetrahydrocannabinol (JWH-255). 2S-methyl-(3,5-dihydroxyphenyl)pentane is prepared in several steps in an enantioselective synthesis from an L-valine derived oxazolidinone of propionic acid and 3,5-dimethoxybenzyl bromide.

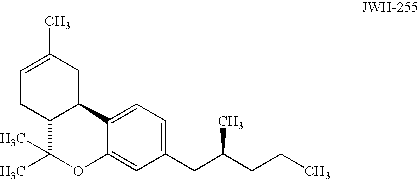

1-Methoxy-3-(2′R-methylhexyl)-Δ⁸-tetrahydrocannabinol, JWH-356, shown below, is prepared from 2R-methyl(3,5-dihydroxyphenyl)hexane and trans-menthadienol to give 3-(2′R-methylhexyl)-Δ⁸-tetrahydrocannabinol. Conversion to the methyl ether is effected by reaction with methyl iodide and potassium hydroxide to provide 1-methoxy-3-(2′R-methylhexyl)-Δ⁸-tetrahydrocannabinol (JWH-356). 2R-methyl-(3,5-dihydroxyphenyl)hexane is prepared in several steps in an enantioselective synthesis from a D-valine derived oxazolidinone of propionic acid and 3,5-dimethoxybenzyl bromide.

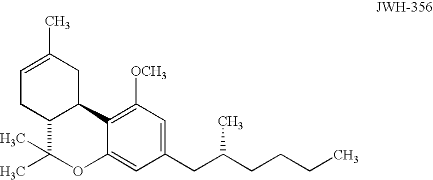

### Example 3

### Binding Affinity of Compounds for CB2 and CB1

Receptor Binding Assays

1. CB₁ Assay. [³H] CP-55,940 (KD=690 nM) binding to P2 membranes was conducted as described elsewhere (Martin, B. R.; Compton, D. R.; Thomas, B. F.; Prescott, W. R., Little; P. J., Razdan, R. K.; Johnson, M. R.; Melvin, L. S.; Mechoulam, R.; Ward, S. J. Pharmacol. Biochem. Behav. 1991, 40, 471.) except whole brain (rather than cortex only) was used. Displacement curves were generated by incubating drugs with 1 nM of [³H] CP-55,940. The assays were performed in triplicate, and the results represent the combined data from three individual experiments.

2. CB₂ Assay. Human embryonic kidney 293 cells were maintained in Dulbecco's modified Eagle's medium (DMEM) with 10% fetal clone II (HyClone, Logan Utah) and 5% CO₂ at 37° C. in a Forma incubator. Cell lines were created by transfection of CB₂pcDNA3 into 293 cells by the Lipofectamine reagent (Life Technologies, Gaithersburg, Md.). The human CB₂ cDNA was a gift. Stable transformants were selected in growth medium containing geneticin (1 mg/mL, reagent, Life Technologies, Gaithersburg, Md.). Colonies of about 500 cells were picked (about 2 weeks post transfection) and allowed to expand, then tested for expression of receptor mRNA by northern blot analysis. Cell lines containing moderate to high levels of receptor mRNA were tested for receptor binding properties. Transfected cell lines were maintained in DMEM with 10% fetal clone II plus 0.3-0.5 mg/mL geneticin and 5% CO₂ at 37° C. in a Forma incubator.

The current assay is a modification of Compton et al. (Compton, D. R.; Rice, K. C.; De Costa, B. R.; Razdan, R. K.; Melvin, L. S.; Johnson, M. R.; Martin, B. R. J. Pharmacol Exp. Ther. 1993, 265, 218.) Cells were harvested in phosphate-buffered saline containing 1 mM EDTA and centrifuged at 500 g. The cell pellet was homogenized in 10 mL of solution A (50 mM Tris-HCl, 320 mM sucrose, 2 mM EDTA, 5 mM MgCl₂, pH 7.4). The homogenate was centrifuged at 1,600×g (10 min), the supernatant saved, and the pellet washed three times in solution A with subsequent centrifugation. The combined supernatants were centrifuged at 100,000×g (60 min). The (P2 membrane) pellet was resuspended in 3 mL of buffer B (50 mM Tris-HCl, 1 mM EDTA, 3 mM MgCl₂, pH 7.4) to yield a protein concentration of approximately 1 mg/mL. The tissue preparation was divided into equal aliquots, frozen on dry ice, and stored at −70° C. Binding was initiated by the addition of 40-50 μg membrane protein to silanized tubes containing [³H]CP-55,940 (102.9 Ci/mmol) and a sufficient volume of buffer C (50 mM Tris-HCl, 1 mM EDTA, 3 mM MgCl₂, and 5 mg/mL fatty acid free BSA, pH 7.4) to bring the total volume to 0.5 mL. The addition of 1 μM unlabelled CP-55,940 was used to assess nonspecific binding. Following incubation (30° C. for 1 hour), binding was terminated by the addition of 2 mL of ice cold buffer D (50 mM Tris-HCl, pH 7.4, plus 1 mg/mL BSA) and rapid vacuum filtration through Whatman GF/C filters (pretreated with polyethyleneimine (0.1%) for at least 2 hours). Tubes were rinsed with 2 mL of ice cold buffer D, which was also filtered, and the filters subsequently rinsed twice with 4 mL of ice cold buffer D. Before radioactivity was quantitated by liquid scintillation spectrometry, filters were shaken for 1 hr in 5 mL of scintillation fluid.

CP-55,940 and all cannabinoid analogs were prepared by suspension in assay buffer from a 1 mg/mL ethanolic stock without evaporation of the ethanol (final concentration of no more than 0.4%). Competition assays were conducted with 1 nM [³H]-CP55,940 and 6 concentrations (0.1 nM to 10 μM displacing ligands). Displacement IC₅₀ values were originally determined by unweighted least-squares linear regression of log concentration-percent displacement data and then converted to Ki values using the method of Cheng and Prusoff(Cheng, Y. C.; Prusoff, W. H. Biochem. Pharmacol. 1973, 22, 3099.)

The results for representative compounds of generic Formula 1 are presented in Table 1.

TABLE 1

| | | | Fold selectivity for |
|---|---|---|---|
| Compound | CB₂ affinity (nM) | CB₁ affinity (nM) | CB₂ over CB₁ |
| 299 | 29.75 ± 1.77 | 415 ± 49.8 | 14 |
| 310 | 36.1 ± 2.8 | 1059 | 29 |
| 349 | 38 ± 4.3 | 375.7 ± 0.7 | 10 |
| 350 | 11.8 ± 1.03 | 395 ± 19.79 | 33 |

The results for representative compounds of generic Formula 2 are presented in Table 2.

| | | | Fold selectivity for |
|---|---|---|---|
| Compound | CB₂ affinity (nM) | CB₁ affinity (nM) | CB₂ over CB₁ |
| 359 | 13.3 ± 0.15 | 2918 ± 450 | 219 |
| 352 | 46.53 ± 2.16 | >10,000 | 213 |
| 353 | 31.3 ± 0.78 | 1493 ± 10 | 48 |
| 255 | 24.33 ± 8.6 | 4307 ± 649 | 174 |
| 356 | 48.4 ± 1.5 | 5837 ± 701 | 56 |

All of the compounds have high affinity for CB₂ receptors and low affinity for CB₁ receptors. CB₂ selectivity ranges from 10 to more than 200 fold.

### Example 4

### Inhibition of Inflammatory Pain using Compounds of the Invention

There have been several reports demonstrating that CB₂ selective agonists are effective in inflammatory pain models (Malan et al., 2001, Clayton et al., 2002, Malan et al., 2002, Quartilho et al., 2003). One of the above CB2 selective agonists was chosen for testing in the formalin model of inflammatory pain in mice. Subjects consisted of male ICR mice (Harlan Labs) weighing between 20-25 g. The formalin test was carried out in an open Plexiglas cage, with a mirror placed under the floor to allow an unobstructed view of the paws. Mice were allowed to acclimate for 15 min in the test cage before formalin injection. Each animal was injected with 20 μl of 2.5% formalin in the intraplantar region of the right hindpaw. Mice were then observed 0-5 min (Phase 1) and 20-45 min (Phase 2) post-formalin, and the amount of time spent licking the injected paw was recorded. The drug was injected i.p. 30 min before formalin injection.

The results are given in FIG. 1 and demonstrate that JWH-350 inhibits nociception in a dose-dependent manner with a dose of 20 mg/kg completely eliminating the pain response. These data demonstrate that a high affinity CB2 selective agonist that is representative of these two series of compounds is an effective analgesic agent in a model of inflammatory pain.

### References for Example 4

Clayton N., Marshall F. H., Bountra C., O'Shaughnessy C. T., 2002. CB1 and CB2 cannabinoid receptors are implicated in inflammatory pain. 96, 253-260.
Malan T. P., Ibrahim M. M., Vanderah T. W., Malkriyannis A., Porreca F., 2002. Inhibition of pain responses by activation of CB(2) cannabinoid receptors. Chemistry and Physics of Lipids 121, 191-200.
Malan T. P., Jr., Ibrahim M. M., Deng H., Liu Q., Mata H. P., Vanderah T., Porreca F., Makriyannis A., 2001. CB2 cannabinoid receptor-mediated peripheral antinociception. 93, 239-245.
Quartilho A., Mata H. P., Ibrahim M. M., Vanderah T. W., Porreca F., Makriyannis A., Malan T. P., Jr., 2003. Inhibition of inflammatory hyperalgesia by activation of peripheral CB2 cannabinoid receptors. Anesthesiology 99, 955-960.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A compound of general formula 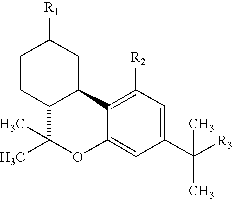
wherein
R₁ is OH and may be in either the α or β configuration;
R₂ may be H or OCH₃; and
R₃ is an alkyl chain from 2 to 6 carbon atoms in length.

2. The compound of claim 1, wherein said compound is

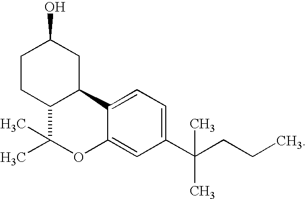

3. The compound of claim 1, wherein said compound is

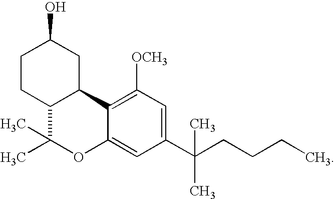

4. The compound of claim 1, wherein said compound is

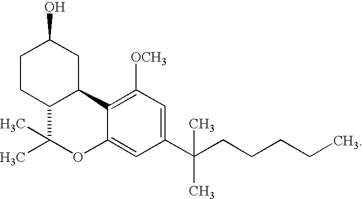

5. The compound of claim 1, wherein said compound is

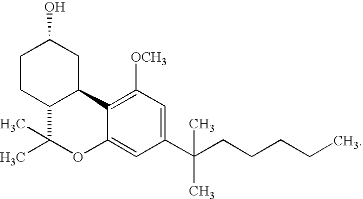

6. A compound of general formula 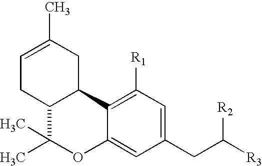
wherein
R¹ is H or OCH₃;
R₂ is CH₃ and is in either the R or S configuration; and
R₃ is an alkyl chain from 2 to 6 carbon atoms in length.

7. The compound of claim 6, wherein said compound is

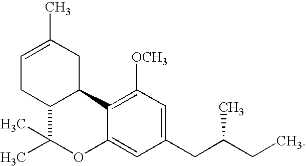

8. The compound of claim 6, wherein said compound is

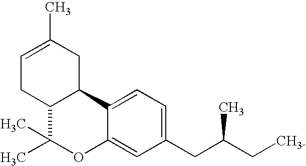

9. The compound of claim 6, wherein said compound is

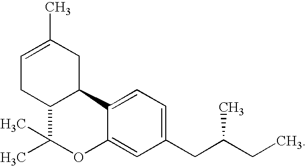

10. The compound of claim 6, wherein said compound is

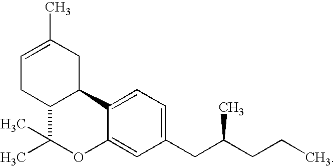

11. The compound of claim 6, wherein said compound is

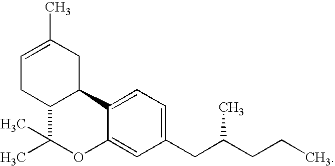

12. The compound of claim 6, wherein said compound is

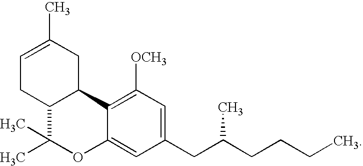

13. A method for selectively binding to CB₂ receptors and not to CB₁ receptors, comprising the step of
exposing said CB₂ receptors and said CB₁ receptors to a compound of general formula 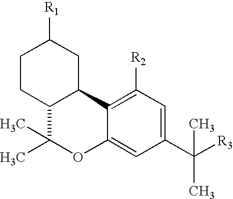
wherein
R₁ is OH and may be in either the α or β configuration;
R₂ may be H or OCH₃; and
R₃ is an alkyl chain from 2 to 6 carbon atoms in length.

14. The method of claim 13, wherein said compound is

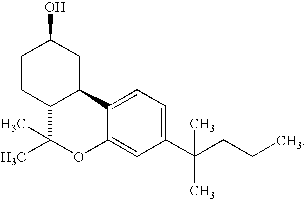

15. The method of claim 13, wherein said compound is

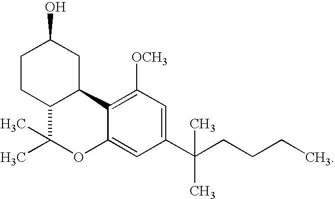

16. The method of claim 13, wherein said compound is

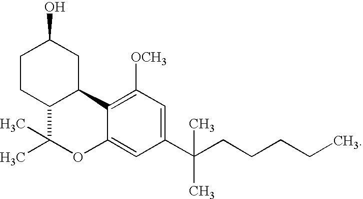

17. The method of claim 13, wherein said compound is

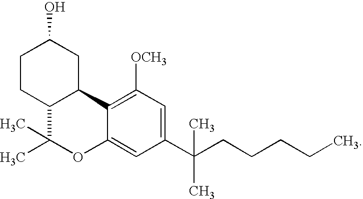

18. The method of claim 13, wherein said compound binds to said CB₂ receptors in an amount sufficient to treat cancer.

19. The method of claim 13 wherein said compound binds to said CB₂ receptors in an amount sufficient to treat pain.

20. The method of claim 13, wherein said compound binds to said CB₂ receptors in an amount sufficient to treat inflammation.

21. A method for selectively binding to CB₂ receptors and not to CB₁ receptors, comprising the step of
exposing said CB₂ receptors and said CB₁ receptors to a compound of general formula 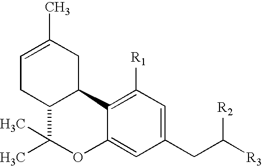
wherein
R₁ is H or OCH₃;
R₂ is CH₃ and is in either the R or S configuration; and
R₃ is an alkyl chain from 2 to 6 carbon atoms in length.

22. The method of claim 21, wherein said compound is

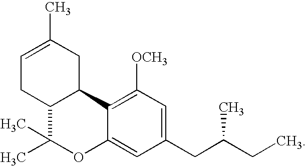

23. The method of claim 21, wherein said compound is

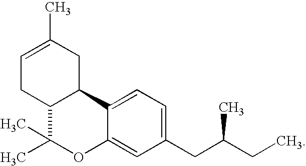

24. The method of claim 21, wherein said compound is

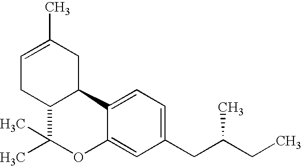

25. The method of claim 21, wherein said compound is

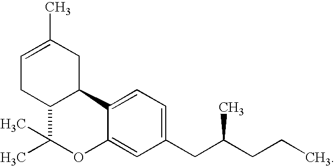

26. The method of claim 21, wherein said compound is

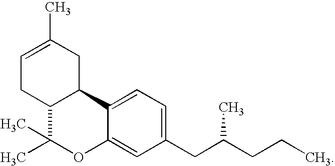

27. The method of claim 21, wherein said compound is

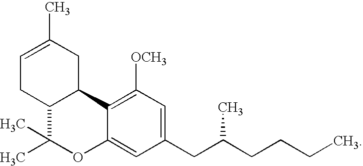

28. The method of claim 21 wherein said compound binds to said CB₂ receptors in an amount sufficient to treat cancer.

29. The method of claim 21 wherein said compound binds to said CB₂ receptors in an amount sufficient to treat pain.

30. The method of claim 21, wherein said compound binds to said CB₂ receptors in an amount sufficient to treat inflammation.
